(19) United States
(12) Patent Application Publication
(10) Pub. No.: US 2014/0315812 A1
(43) Pub. Date: Oct. 23, 2014

(54) AMORPHOUS EZATIOSTAT ANSOLVATE

(71) Applicant: Telik, Inc., Palo Alto, CA (US)

(72) Inventor: Steven R. Schow, Palo Alto, CA (US)

(21) Appl. No.: 13/812,474

(22) PCT Filed: Nov. 30, 2012
(86) PCT No.: PCT/US2012/067352

§ 371 Date: Jan. 25, 2013

Related U.S. Application Data
(60) Provisional application No. 61/566,454, filed on Dec. 2, 2011.
(60) Provisional application No. 61/619,286, filed on Apr. 2, 2012.

(51) Int. Cl. C07K 5/065

(52) U.S. Cl. CPC C07K 5/06078
USPC 514/13.5; 560/16; 514/21.91; 514/19.6; 514/19.3

(57) ABSTRACT

Provided herein is an amorphous form of a pharmaceutically acceptable salt of ezatiostat, for example, ezatiostat hydrochloride, compositions, uses and methods of preparation thereof.

### CROSS-REFERENCE TO RELATED APPLICATIONS

This application claims the benefit under 35 U.S.C. §119(e) of U.S. Provisional Application Nos. 61/566,454, filed Dec. 2, 2011, and 61/619,286, filed Apr. 2, 2012, both of which are incorporated herein by reference in their entirety.

### BACKGROUND

Ezatiostat, also known as TLK199 or TER 199, is a compound of the formula:

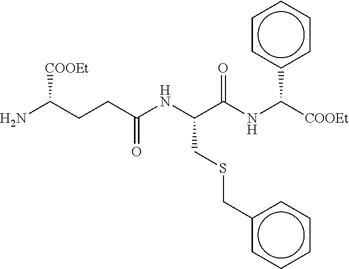

Ezatiostat has been shown to induce the differentiation of HL-60 promyelocytic leukemia cells in vitro, to potentiate the activity of cytotoxic agents both in vitro and in vivo, and to stimulate colony formation of all three lineages of hematopoietic progenitor cells in normal human peripheral blood. In preclinical testing, ezatiostat has been shown to increase white blood cell production in normal animals, as well as in animals in which white blood cells were depleted by treatment with cisplatin or fluorouracil. Similar effects may provide a new approach to treating myelodysplastic syndrome (MDS).

Many conditions, including MDS, a form of pre-leukemia in which the bone marrow produces insufficient levels of one or more of the three major blood elements (white blood cells, red blood cells, and platelets), are characterized by depleted bone marrow. Myelosuppression, which is characterized by a reduction in blood cell levels and in a reduction of new blood cell generation in the bone marrow, is also a common, toxic effect of many standard chemotherapeutic drugs.

Ezatiostat hydrochloride is the hydrochloride acid addition salt of ezatiostat. Ezatiostat hydrochloride in a liposomal injectable formulation was studied in a clinical trial for the treatment of MDS, and results from this trial, reported by Raza et al., J. Hem. Onc., 2:20 (published online 13 May 2009), demonstrated that administration of TLK199 was well tolerated and resulted in multi-lineage hematologic improvement. Ezatiostat hydrochloride in a tablet formulation has been evaluated in a clinical trial for the treatment of MDS, as reported by Raza et al., Blood, 113:6533-6540 (prepublished online 27 Apr. 2009) and a single-patient report by Quddus et al., J. Hem. Onc., 3:16 (published online 23 Apr. 2010), and is currently being evaluated in clinical trials for the treatment of MDS and for severe chronic idiopathic neutropenia.

### SUMMARY

This invention is directed to the amorphous forms of pharmaceutically acceptable salts of ezatiostat which have adequate stability and solubility in a pharmaceutical composition form. Accordingly, in one of its compound aspects, there is provided an amorphous form of a pharmaceutically acceptable salt of ezatiostat. In one embodiment, the amorphous form is an ansolvate. In another embodiment, the pharmaceutically acceptable salt is the hydrochloride salt.

In one of its composition aspects, there is provided a composition comprising a pharmaceutically acceptable excipient and an amorphous form of a pharmaceutically acceptable salt of ezatiostat, for example, the amorphous form of the ansolvate. In one embodiment, the amorphous form of ezatiostat is an ansolvate of ezatiostat hydrochloride. In one embodiment, the amorphous form of ezatiostat hydrochloride is stable for at least about 6 months or at least about 8 months.

In another aspect, provided herein is a method of preparing an amorphous form of a pharmaceutically acceptable salt of ezatiostat. In one embodiment, the method comprises dissolving the salt in a suitable solvent to form a solution followed by flash evaporation. In some embodiments, the solvent is a polar aprotic solvent, such as methylene chloride, tetrahydrofuran, acetone, ethyl acetate, dioxane, chloroform, or a mixture thereof. In some embodiments, the solvent is a protic solvent, such as C₁₋₃ alcohol (an alcohol comprising 1-3 carbon atoms, or a combination thereof), such as methanol and/or ethanol. In one embodiment, the amorphous form of ezatiostat is the ansolvate of ezatiostat hydrochloride. In another embodiment, the method comprises triturating an oil of a pharmaceutically acceptable salt of ezatiostat with a suitable solvent, such as an ether or a hydrocarbon, to obtain an amorphous solid of the pharmaceutically acceptable salt of ezatiostat. In still another embodiments, the method comprises precipitating a pharmaceutically acceptable salt of ezatiostat from a solution of the pharmaceutically acceptable salt of ezatiostat by addition of an antisolvent, such as an ether or a hydrocarbon, and collecting the solid formed therefrom.

In still another aspect, there are provided methods for inducing differentiation of HL-60 promyelocytic leukemia cells or to potentiate the activity of cytotoxic agents in vitro by contacting the cells with an effective amount of a compound or composition of this invention, or in vivo by administering an effective amount of a compound or composition of this invention to a subject in need thereof.

In still another aspect, there are provided methods to stimulate colony formation of all three lineages of hematopoietic progenitor cells (platelets, europhils, and erythrocytes) in normal human peripheral blood in vitro by contacting a blood sample with an amount of a compound or composition of this invention, or in vivo by administering an amount of a compound or composition of this invention to a subject in need thereof.

In still another aspect, there are provided methods of treating multiple myeloma, a myelodysplastic syndrome, severe chronic idiopathic neutropenia, leukemia or other cancers or conditions that involve cytopenia, chemotherapy induced neutropenia, or thrombocytopenia comprising administering a therapeutically effective amount of a compound or composition of this invention to a patient in need of such treatment.

In some embodiments, there are provided methods of treating severe chronic idiopathic neutropenia, leukemia or other cancers and conditions that involve cytopenia, chemotherapy induced neutropenia, or thrombocytopenia comprising administering a therapeutically effective amount of a compound or composition of this invention to a patient in need of such treatment.

In some embodiments, there are provided methods of treating myelodysplastic syndrome (MDS) comprising administering a therapeutically effective amount of a compound or composition of this invention to a patient in need of such treatment.

In all of such treatments, the dosing of a pharmaceutically acceptable salt of ezatiostat to the treated patient is already disclosed in the art.

### BRIEF DESCRIPTION OF THE DRAWING

FIG. 1 shows the differential scanning calorimetry (DSC) of a crystalline ezatiostat hydrochloride.

FIG. 2 shows the X-ray powder diffraction pattern of the crystalline ezatiostat hydrochloride.

FIG. 3 shows the DSC of an amorphous ezatiostat hydrochloride.

FIG. 4 shows the X-ray powder diffraction pattern of the amorphous ezatiostat hydrochloride.

### DETAILED DESCRIPTION

### Definitions

As used herein, the following terms have the following meanings.

The singular forms “a,” “an,” and “the” and the like include plural referents unless the context clearly dictates otherwise. Thus, for example, reference to “a compound” includes both a single compound and a plurality of different compounds.

The term “about” when used before a numerical designation, e.g., temperature, time, amount, and concentration, including a range, indicates approximations which may vary by ±10%, ±5% or ±1%.

“Administration” refers to introducing an agent into a patient. A therapeutic amount can be administered, which can be determined by the treating physician or the like. An oral route of administration is preferred. The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient. In any event, administration entails delivery to the patient of the drug.

“An amorphous form” refers to a solid that lacks the spatial and/or long-range order characteristic of a crystal.

The “ansolvate” of a salt of ezatiostat is a solid form that is substantially free of solvents. As used above, “substantially free of” and “small amounts,” refers to the presence of solvents preferably less than 10,000 parts per million (ppm), or more preferably, less than 5000 ppm, and still more preferably less than 1,000 ppm or 500 ppm.

“Characterization” refers to obtaining data which may be used to identify a solid form of a compound, for example, to identify whether the solid form is an amorphous or crystalline form and whether it is an ansolvated or solvated form. The process by which solid forms are characterized involves analyzing data collected on the polymorphic forms so as to allow one of ordinary skill in the art to distinguish one solid form from other solid forms containing the same material. Chemical identity of solid forms can often be determined with solution-state techniques such as ¹³C NMR or ¹H NMR. While these may help identify a material, and a solvent molecule for a solvate, such solution-state techniques themselves may not provide information about the solid state. There are, however, solid-state analytical techniques that can be used to provide information about solid-state structure and differentiate among amorphous and polymorphic solid forms, such as X-ray powder diffraction (XRPD), solid state nuclear magnetic resonance (SS-NMR), infrared and Raman spectroscopy, and thermal techniques such as differential scanning calorimetry (DSC), thermogravimetry (TG), melting point, and hot stage microscopy. It is understood by the skilled artisan that data obtained by the above analyses in different experiments may vary depending on the condition and instrument used in the analyses. The data obtained by particular analytic technique with different experiments are “substantially the same” when characteristic data obtained using the same analytic technique (but may be obtained under different conditions or using different instruments) vary within ±10%, ±5% or ±1%. For example, the term “substantially the same” in the context of XRPD is meant that characteristic peaks of the XRPD of a solid material vary within ±10%, ±5% or ±1%. A skilled artisan would recognize characteristic data for each particular analytical technique when presented with data obtained by the analysis. For example, characteristic of data of an XRPD are peaks described below that can distinguish one solid form of a compound from another solid form of the compound, and characteristic data of a differential scanning calorimetry are those relate to the transitional events particular to a solid form.

To “characterize” a solid form of a compound, one may, for example, collect XRPD data on a solid form of the compound and optionally compare the XRPD peaks of with XRPD peaks of other form(s) or of a known standard. For example, when only two solid forms, I and II, are compared and the form I pattern shows a peak at an angle where no peaks appear in the form II pattern, then that peak, for that compound, distinguishes form I from form II and further acts to characterize form I. The collection of peaks which distinguish form I from the other known forms is a collection of peaks which may be used to characterize form I. Those of ordinary skill in the art will recognize that there are often multiple ways, including multiple ways using the same analytical technique, to characterize solid forms. Additional peaks up to and including an entire diffraction pattern could also be used, but are not necessary, to characterize the form. Although all the peaks within an entire XRPD pattern may be used to characterize such a form, a subset of that data may, and typically is, used to characterize the form.

X-ray powder diffraction (XRPD) analyses can be performed on a Shimadzu XRD-6000 X-ray powder diffractometer using Cu Kα radiation from a long fine focus X-ray tube, operated at 40 kV, 40 mA. The divergence and scattering slits can be set at 1° and the receiving slit can be set at 0.15 mm. Diffracted radiation can be detected by a NaI scintillation detector. A θ-2θ continuous scan at 3°/min (0.4 sec/0.02° step) from 2.5°-40° 2θ can be used. A silicon standard can be analyzed to check alignment of the instrument. Data can be collected and analyzed using XRD-6000 v.4.1 software.

Differential scanning calorimetry (DSC) analyses can be performed on a TA Instruments Q100 or 2920 differential scanning calorimeter, which can be calibrated using indium as the reference material. The sample can be placed into a standard aluminum DSC pan with an uncrimped lid, and the weight accurately recorded. The sample cell can be equilibrated at 25° C. and heated under a nitrogen purge at a rate of 10° C./minute to a final temperature of 250° C. The variability of DSC data is affected by sample preparation and particularly by heating rate.

Solid-state NMR (SS-NMR) ¹³C cross-polarization magic angle spinning (CP/MAS) analyses can be performed at room temperature on a Varian ^{UNITY}INOVA-400 spectrometer (Larmor frequencies: ¹³C=100.542 MHz, ¹H=399.800 MHz). The sample can be packed into a 4 mm PENCIL type zirconia rotor and rotated at 12 kHz at the magic angle. The spectrum can be acquired with phase modulated SPINAL-64 high power ¹H decoupling during the acquisition time using a ¹H pulse width of 2.2 μs (90°), a ramped amplitude cross polarization contact time of 2 ms, a 30 ms acquisition time, a 5 second delay between scans, a spectral width of 45 KHz with 2700 data points, and 200 co-added scans. The free induction decay (FID) can be processed using Varian VNMR 6.1C software with 32768 points and an exponential line broadening factor of 10 Hz to improve the signal-to-noise ratio. The first three data points of the FID can be back predicted using the VNMR linear prediction algorithm to produce a flat baseline. The chemical shifts of the spectral peaks can be externally referenced to the carbonyl carbon resonance of glycine at 176.5 ppm. The variability of SS-NMR peaks in this experiment is considered to be ±0.2 ppm.

Karl Fischer analyses for water determination can be performed on a Mettler Toledo DL39 Karl Fischer titrator. About 10-15 mg of sample can be placed in the KF titration vessel containing approximately 100 mL of Hydranal®-Coulomat AD reagent and mixed for 60 seconds to ensure dissolution. The dissolved sample can be then titrated by means of a generator electrode which produces iodine by electrochemical oxidation.

Thermogravimetric (TG-IR) analyses can be performed on a TA Instruments model 2050 thermogravimetric (TG) analyzer interfaced to a Thermo Nicolet Magna® 560 Fourier transform infrared (FT-IR) spectrophotometer equipped with a Ever-Glo mid/far IR source, a potassium bromide beamsplitter, and a deuterated triglycine sulfate detector. The instrument can be operated under a flow of helium at 90 mL/min (purge) and 10 mL/min (balance). The sample can be placed in a platinum sample pan, inserted into the TG furnace, accurately weighed by the instrument, and heated from ambient at a rate of 20° C./min. The TG instrument is started first, immediately followed by the FT-IR instrument. IR spectra can be collected every 12.86 seconds; and each IR spectrum represents 32 co-added scans collected at a spectral resolution of 4 cm⁻¹. A background scan can be collected before the beginning of the experiment. Wavelength calibration can be performed using polystyrene. The TG calibration standards can be nickel and Alumel™.

Hot stage microscopy analysis can be performed on a Linkam FTIR 600 hot stage mounted on a Leica DM LP microscope. Samples can be observed using a 20× objective with cross polarizers and lambda compensator. A coverslip can be then placed over the sample. Each sample can be visually observed as the stage is heated. Images can be captured using a SPOT Insight™ color digital camera with SPOT Software v. 3.5.8. The hot stage can be calibrated using USP melting point standards.

“Comprising” or “comprises” is intended to mean that the compositions and methods include the recited elements, but do not exclude others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Room temperature” refers to (22±5)° C.

“Therapeutically effective amount” or “therapeutic amount” refers to an amount of a drug or an agent that when administered to a patient suffering from a disease, disorder, or a condition, will have the intended therapeutic effect, e.g., alleviation, amelioration, palliation or elimination of one or more manifestations of the condition in the patient. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating myelodysplastic syndrome, refers to an amount of the agent that alleviates, ameliorates, palliates, or eliminates one or more manifestations of the myelodysplastic syndrome in the patient.

“Treatment”, “treating”, and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition. For purposes of this invention, beneficial or desired clinical results include, but are not limited to, multilineage hematologic improvement, decrease in the number of required blood transfusions, decrease in infections, decreased bleeding, and the like.

### Amorphous Form

Provided herein in one aspect is an amorphous form of a pharmaceutically acceptable salt of ezatiostat. In some embodiments, the amorphous form is an ansolvate. In some embodiments, the pharmaceutically acceptable salt of ezatiostat is ezatiostat hydrochloride. In some embodiments, the amorphous form is an amorphous form of ezatiostat hydrochloride ansolvate. In some embodiments, the amorphous form has an XRPD pattern that does not have any distinct peak. A distinct peak refers to a peak whose width at baseline is no more than 10° 2θ or no more than 5° 2θ. In some embodiments, the amorphous form has an XRPD pattern that comprises a broad peak whose width at baseline is at least 5° 2θ or at least 10° 2θ. In one embodiment, the amorphous form of ezatiostat hydrochloride is stable for at least 6 months or at least 8 months, such as at a low temperature, e.g., 0-10° C. or 0-5° C.

### Preparation

An amorphous form of a pharmaceutically acceptable salt of ezatiostat can be prepared by dissolving the pharmaceutically acceptable salt of ezatiostat, for example, a hydrochloride salt, in a suitable solvent to form a solution followed by flash evaporation to prevent crystallization. In some embodiments, the solvent is a polar aprotic solvent, such as methylene chloride, tetrahydrofuran, acetone, ethyl acetate, dioxane, chloroform, or a mixture thereof. In some embodiments, the solvent is C₁₋₃ alcohol, such as methanol or ethanol. Alternatively, the amorphous form can be prepared by triturating an oil of a pharmaceutically acceptable salt of ezatiostat such as an oil obtained by evaporating a solution of the pharmaceutically acceptable salt of ezatiostat, with a suitable solvent, such as an ether or a hydrocarbon, to obtain an amorphous solid of the pharmaceutically acceptable salt of ezatiostat. Examples of ether include dimethyl ether, diethyl ether and methyl tert-butyl ether, etc. Examples of hydrocarbon include benzene, hexane, and toluene, etc. An amorphous form of a pharmaceutically acceptable salt of ezatiostat may also be prepared by adding an antisolvent, such as an ether or a hydrocarbon, to a solution of the pharmaceutically acceptable salt of ezatiostat, such as that described above, to precipitate the pharmaceutically acceptable salt of ezatiostat. The resulting solid by either trituration or precipitation can be collected by, for example, filtration or decantation and drying.

### Composition

In another aspect, provided herein is a pharmaceutical composition comprising the amorphous form of a pharmaceutically acceptable salt of ezatiostat provided herein and a pharmaceutically acceptable excipient. In some embodiments, the pharmaceutical composition is in an oral form. In some embodiments, the pharmaceutical composition is in an injectable form. In one embodiment, the pharmaceutical composition is in a lipid formulation as described in U.S. Pat. No. 7,029,695. In another embodiment, the pharmaceutical composition is in a tablet formulation, such as the tablet formulation disclosed in U.S. Patent Application Publication US 2011/0300215 A1, filed Mar. 29, 2011, titled “TABLET FORMULATION OF EZATIOSTAT,” which is incorporated by reference in its entirety.

### Treatment Methods

In another aspect, provided herein is a method of treating severe chronic idiopathic neutropenia, leukemia or other cancers and conditions that involve cytopenia, chemotherapy induced neutropenia, or thrombocytopenia comprising administering an amount of an amorphous form of a pharmaceutically acceptable salt of ezatiostat provided herein to a patient in need of such treatment.

In another aspect, provided herein is a method of treating multiple myeloma comprising administering an amount of an amorphous form of a pharmaceutically acceptable salt of ezatiostat provided herein to a patient in need of such treatment.

In another aspect, provided herein is a method of treating a myelodysplastic syndrome comprising administering an amount of an amorphous form of a pharmaceutically acceptable salt of ezatiostat provided herein to a patient in need of such treatment.

Certain methods of therapeutic uses of ezatiostat are further described in U.S. Patent Application Publication Nos. US 2011/0301102 A1, US 2011/0301198 A1, and US 2011/0301199 A1, and US 2012/0251496, and U.S. patent application Ser. No. 13/437,474, filed on Apr. 2, 2012, titled “METHODS FOR TREATING MYELODYSPLASTIC SYNDROME WITH EZATIOSTAT,” the contents of all of which are incorporated herein by reference in their entirety.

In some embodiments, the amount is an effective amount. In some embodiments, the effective amount is selected from (when calculated in terms of the amount of ezatiostat hydrochloride):
1.5 gram of the amorphous form of ezatiostat hydrochloride administered twice per day for 2 weeks for an aggregate total dosing of 42 grams followed by a week when no ezatiostat or a salt is administered;
1 gram of the amorphous form of ezatiostat hydrochloride administered twice per day for 3 weeks for an aggregate total dosing of 42 grams followed by a week when no ezatiostat or a salt is administered;
1 gram of the amorphous form of ezatiostat hydrochloride administered twice per day continuously until the attending clinician deems it appropriate for the patient to be withdrawn from administration;
a therapeutically effective amount of up to 3 grams of the amorphous form of ezatiostat hydrochloride per day administered in one, two, or three divided doses for 2 weeks followed by a week when no ezatiostat or a salt is administered;
a therapeutically effective amount of up to 2 grams of the amorphous form of ezatiostat hydrochloride per day administered in one, two, or three divided doses for 3 weeks followed by a week when no ezatiostat or a salt is administered; and/or
a therapeutically effective amount of up to 2 grams of the amorphous form of ezatiostat hydrochloride per day administered in one, two, or three divided doses continuously until the attending clinician deems it appropriate for the patient to be withdrawn from administration.

Although the above amounts are described in terms of the amount of ezatiostat hydrochloride, a person skilled in the art will appreciate that an amorphous form of other pharmaceutically acceptable salts of ezatiostat can be administered in an amount that provides an equivalent amount of ezatiostat to any of the amounts of ezatiostat hydrochloride described above.

In some embodiments, the amorphous form of a pharmaceutically acceptable salt of ezatiostat is administered with lenalidomide. In some embodiments, the amorphous form of a pharmaceutically acceptable salt of ezatiostat is administered to a MDS patient having prior exposure to lenalidomide.

In some embodiments, the amorphous form of a pharmaceutically acceptable salt of ezatiostat is administered to a MDS patient having prior exposure to a DNA methyltransferase inhibitor and is administered with lenalidomide or after administration of lenalidomide.

### EXAMPLES

### Example 1

80 mg of crystalline ezatiostat hydrochloride was placed in a round bottom flask and dissolved in 25 mL of methanol. The solvent was then evaporated on a rotary evaporation apparatus under reduced pressure at 30° C. After 30 minutes, the solid sample was removed from the round bottom flask and stored in a sealed vial at 2° C. in a refrigerator. Analysis of this sample was carried out within 24 hours of removing it from the rotary evaporation apparatus.

The resulting amorphous material was analyzed by ¹H NMR, ¹³C NMR, DSC, and X-Ray powder diffraction experiments. The DSC conditions were 30 to 300° C. at 10° C./min using 7 mg of the amorphous material. The X-Ray powder diffraction was taken at 0-60 of 2theta. The crystalline ezatiostat hydrochloride was also analyzed.

The ¹H and ¹³C NMR spectra for crystalline and amorphous ezatiostat hydrochloride match well indicating that there was no degradation during the above process. The DSC and X-ray powder diffraction data, however, are quite different.

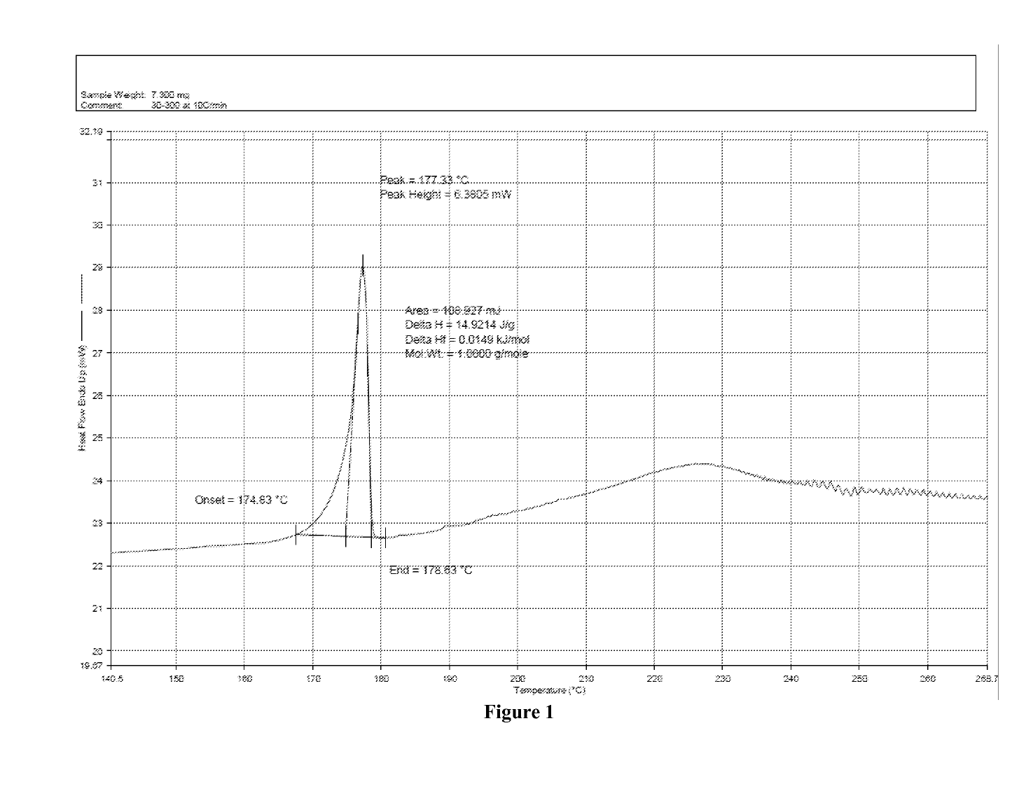

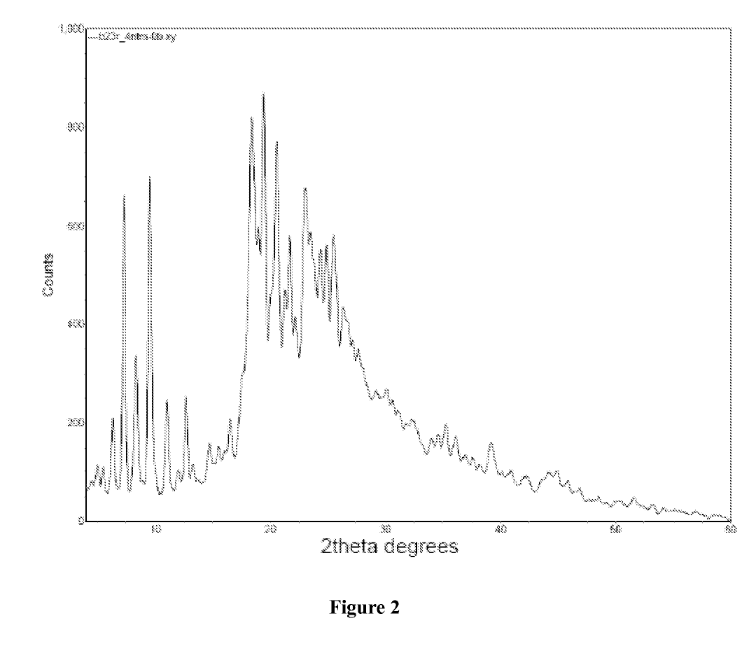

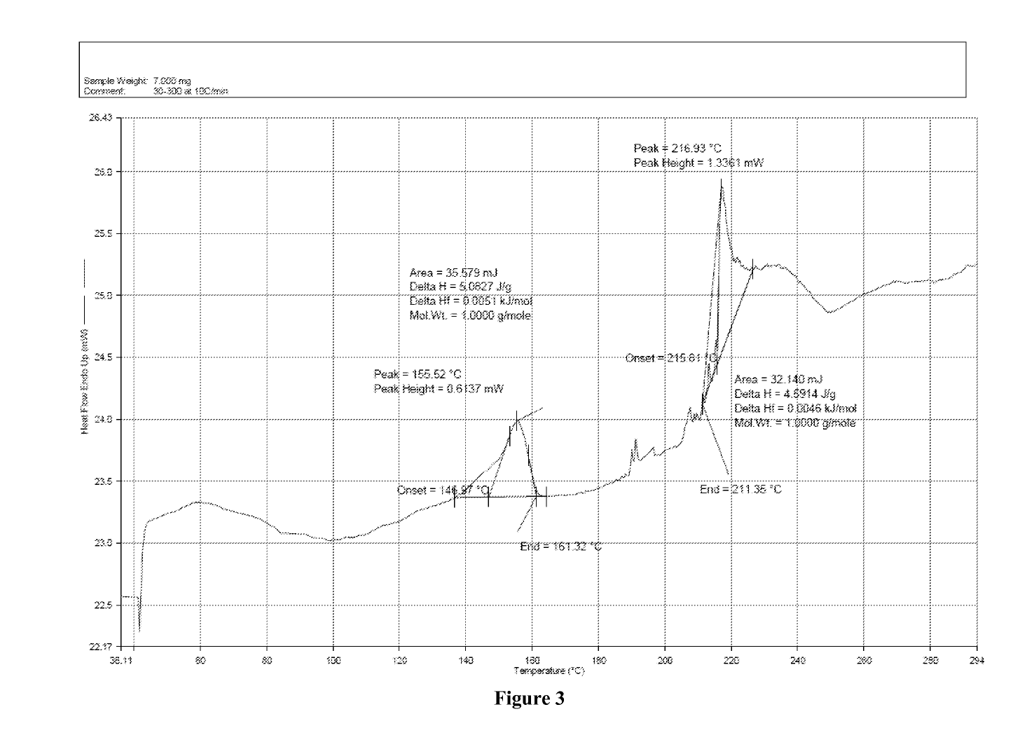

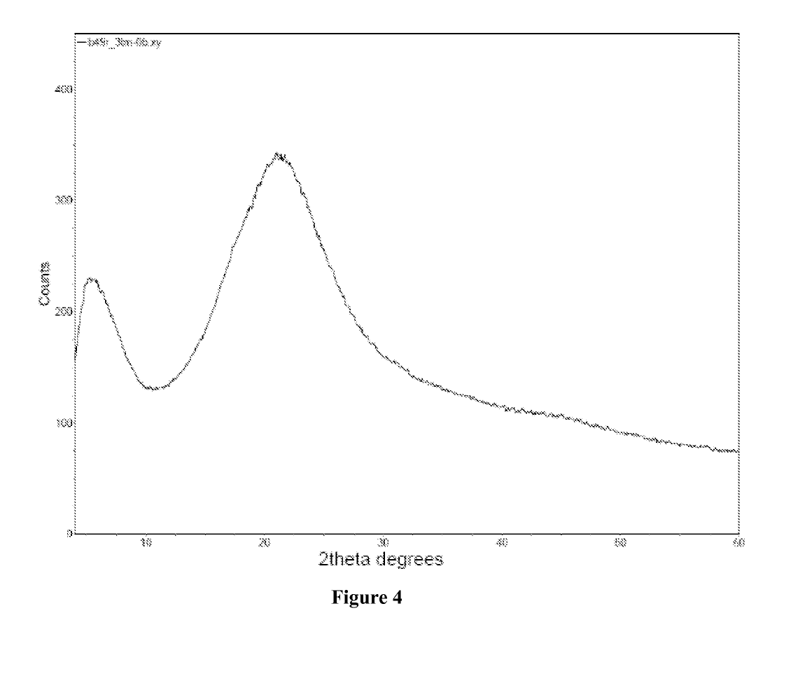

The differential scanning calorimetry (DSC) and X-ray powder diffraction pattern, respectively, of the crystalline ezatiostat hydrochloride are shown in FIGS. 1 and 2. The DSC and powder diffraction pattern of the amorphous ezatiostat hydrochloride, respectively, are shown in FIGS. 3 and 4.

The DSC of the crystalline material shows a flat baseline and a single sharp peak for the melting point phase transition at about 177° C. (FIG. 1). The DSC of amorphous material shows a step-like incline and additional signals associated with transitional events (FIG. 3), including one at about 156° C. and one at about 217° C., indicating that the material is amorphous.

The crystalline material showed sharp peaks in the X-ray powder diffraction pattern (FIG. 2). The amorphous material has an X-ray powder diffraction pattern having broad or widened peaks (FIG. 4) indicating that it is amorphous.

### Example 2

The stability of the amorphous form was evaluated by visual inspection of a sample of the amorphous form prepared above after being stored at about 0-5° C. for about 8 months. No change with respect to color, texture and flowability of the sample and no crystal formation was observed by the visual inspection, indicating that the amorphous form is stable for at least about 8 months under 0-5° C.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed is:

1. A pharmaceutically acceptable salt of amorphous ezatiostat.

2. A pharmaceutically acceptable salt of amorphous ezatiostat, which is an ansolvate.

3. A pharmaceutically acceptable salt of amorphous ezatiostat, which is ezatiostat hydrochloride exhibiting stability of at least 8 months.

4. The pharmaceutically acceptable salt of amorphous ezatiostat of claim 3, which is an ansolvate.

5. The pharmaceutically acceptable salt of amorphous ezatiostat of claim 3, having an X-ray powder diffraction pattern that is substantially the same as FIG. 4.

6. The pharmaceutically acceptable salt of amorphous ezatiostat of claim 3, having a differential scanning calorimetry that is substantially the same as FIG. 3.

7. A composition comprising the pharmaceutically acceptable salt of amorphous ezatiostat of any one of claims 1-6.

8. A method of treating severe chronic idiopathic neutropenia comprising administering a therapeutically effective amount of the pharmaceutically acceptable salt of amorphous ezatiostat of any one of claims 1-6, or the composition of claim 7 to a patient in need of such treatment.

9. A method of treating leukemia comprising administering a therapeutically effective amount of the pharmaceutically acceptable salt of amorphous ezatiostat of any one of claims 1-6, or the composition of claim 7 to a patient in need of such treatment.

10. A method of treating multiple myeloma comprising administering a therapeutically effective amount of the pharmaceutically acceptable salt of amorphous ezatiostat of any one of claims 1-6, or the composition of claim 7 to a patient in need of such treatment.

11. A method of treating a myelodysplastic syndrome comprising administering a therapeutically effective amount of the pharmaceutically acceptable salt of amorphous ezatiostat of any one of claims 1-6, or the composition of claim 7 to a patient in need of such treatment.

12. A method of treating chemotherapy induced neutropenia comprising administering a therapeutically effective amount of the pharmaceutically acceptable salt of amorphous ezatiostat of any one of claims 1-6, or the composition of claim 7 to a patient in need of such treatment.

13. A method of treating thrombocytopenia comprising administering a therapeutically effective amount of the pharmaceutically acceptable salt of amorphous ezatiostat of any one of claims 1-6, or the composition of claim 7 to a patient in need of such treatment.

14. A method of treating a cancer, said method comprising administering a therapeutically effective amount of the pharmaceutically acceptable salt of amorphous ezatiostat of any one of claims 1-6, or the composition of claim 7 to a patient in need of such treatment.

15. A method of treating a condition that involve cytopenia, said method comprising administering a therapeutically effective amount of the pharmaceutically acceptable salt of amorphous ezatiostat of any one of claims 1-6, or the composition of claim 7 to a patient in need of such treatment.
